(19) United States
(12) Patent Application Publication
(10) Pub. No.: US 2019/0069849 A1
(43) Pub. Date: Mar. 7, 2019

(54) COMPRESSED SENSING ENABLED SWEPT SOURCE OPTICAL COHERENCE TOMOGRAPHY APPARATUS, COMPUTER-ACCESSIBLE MEDIUM, SYSTEM AND METHOD FOR USE THEREOF

(71) Applicant: THE TRUSTEES OF COLUMBIA UNIVERSITY IN THE CITY OF NEW YORK, New York, NY (US)

(72) Inventors: Christine Hendon, Long Island City, NY (US); Yuye Ling, Shanghai (CN)

(21) Appl. No.: 16/120,891

(22) Filed: Sep. 4, 2018

Related U.S. Application Data
(60) Provisional application No. 62/553,472, filed on Sep. 1, 2017.
(60) Provisional application No. 62/699,792, filed on Jul. 18, 2018.

(51) Int. Cl. A61B 5/00; G06T 11/00

(52) U.S. Cl. CPC A61B 5/7232; A61B 5/0066; A61B 2576/00; A61B 2560/0223; G06T 11/005

(57) ABSTRACT

An exemplary system, method and computer-accessible medium for compressing data that can be based on an optical coherence tomography (OCT) signal can be provided, which can include, for example, receiving OCT data from a digital acquisition board that can be based on the OCT signal, storing the OCT data in a volatile memory, and compressing the stored OCT data using a compressed sensing procedure. The compressed sensing procedure can be based on a software mask residing on the computer hardware arrangement. The stored OCT data can be compressed using the software mask to mask particular portions of the stored OCT data. The compressed OCT data can be stored in a non-volatile data storage arrangement. The OCT signal can be an OCT calibration signal.

### CROSS-REFERENCE TO RELATED APPLICATION(S)

This application relates to U.S. Patent Application Nos. 62/553,472, filed on Sep. 1, 2017, and 62/699,792, filed on Jul. 18, 2018, the entire disclosures of which are incorporated herein by reference.

### STATEMENT REGARDING FEDERALLY SPONSORED RESEARCH

This invention was made with government support under grant HL127776 awarded by the National Institutes of Health and 1454365 awarded by the National Science Foundation. The government has certain rights in the invention.

### FIELD OF THE DISCLOSURE

The present disclosure relates generally to an optical coherence tomography (“OCT”) system, and more specifically, to exemplary embodiments of an exemplary compressed-sensing (“CS”) enabled swept-source (“SS”) OCT apparatus and method for use thereof.

### BACKGROUND INFORMATION

Recent advancements in laser technologies have changed the landscape of swept source optical coherence tomography (“SS-OCT”); the A-line rate of a wavelength swept laser source has been improved dramatically from about 2 kHz to about 28 MHz in the past decades. (See e.g., References 1 and 2). This unprecedented scanning speed has enabled numerous exciting real-time applications. (See, e.g., References 3-5). However, the resulting large amount of data to be transferred, processed, and recorded has become a big challenge for engineers. For example, a moderate 200 kHz SS-OCT system could generate about 800 MB of data every second, if each A-line is digitized by 2000 points at a 12-bit precision. Therefore, the mismatch between how much data is generated from a modern SS-OCT and how much data can be effectively transferred and recorded using off-the-shelf electronics can be significant. In fact, the data transfer and record rate has been recognized as the main constraint in current SS-OCT systems. (See, e.g., Reference 3). This is even worse for functional SS-OCT systems, where multiple channels are generally recorded. For example, two channels of signals with orthogonal polarization states are usually recorded in polarization-sensitive OCT. In phase-resolved SS-OCT, a simultaneously recorded reference clock channel (see e.g., Reference 6), or an oversampling procedure (see e.g., Reference 7), is often applied to stabilize the phase of the measurement. A dual-channel 200 kHz SS-OCT system over-sampled at 2 GS/s could generate a data rate of 8 GB/s.

Thus, it may be beneficial to provide an exemplary CS-enabled SS-OCT apparatus and method which can overcome at least some of the deficiencies described herein above.

### SUMMARY OF EXEMPLARY EMBODIMENTS

An exemplary system, method and computer-accessible medium for compressing data that can be based on an optical coherence tomography (“OCT”) signal can be provided, which can include, for example, receiving OCT data from a digital acquisition (“DAQ”) board(s) that can be based on the OCT signal, storing the OCT data in a volatile memory, and compressing the stored OCT data using a compressed sensing procedure. The compressed sensing procedure can be based on a software mask residing on the computer hardware arrangement. The stored OCT data can be compressed using the software mask to mask particular portions of the stored OCT data. The compressed OCT data can be stored in a non-volatile data storage arrangement. The OCT signal can be an OCT calibration signal.

In some exemplary embodiments of the present disclosure, the OCT data can be reconstructed using the CS procedure. An analog signal related to the OCT data can be received and the OCT data can be generated using the DAQ board(s). The stored OCT data can be compressed by down-sampling the OCT data using the CS procedure. The CS procedure can be based on a binary mask having a particular compression ratio. The binary mask can be randomly generated. The OCT data can be generated by fully digitizing a sample channel and a clock channel from an OCT scan at a full rate. The sample channel can be fully digitized using a first DAQ Board, and the clock channel can be fully digitized using a second DAQ board, where the first DAQ board can be different from the second DAQ board. The OCT data can be generated by digitizing an OCT signal related to the OCT data using a plurality of registers, where each of the registers can specify a trigger event to ignore a portion of the OCT signal during digitization.

Further, an exemplary digital acquisition (“DAQ”) board for use in an optical coherence tomography (“OCT”) system, can be provided, which can include, for example, at least one hardware signal mask configured to receive an OCT signal and mask particular portions of the OCT signal to generate a masked OCT signal, and an analog to digital (“A/D”) converter(s) receiving and converting the masked OCT signal into a digital format. The OCT signal can be an OCT calibration signal. The hardware signal mask(s) can be configured to mask the particular portions based on a compressed sensing procedure. The hardware signal mask(s) can be configured to mask the particular portions using a plurality of registers, where each of the registers can specify a trigger event to ignore a portion(s) of the OCT signal during digitization

A method for compressing an optical coherence tomography (“OCT”) signal, can be provided, which can include, for example, generating first and second OCT signals based on the OCT signal, storing the first OCT signal in memory as stored data, and masking particular portions of the stored data using a first compressed sensing (“CS”) procedure thereby generating first OCT information. Second OCT information can be generated by masking particular portions of the second OCT signal based on a second CS procedure, and digitizing unmasked portions of the second OCT signal. The compressed OCT signal can then be generated based on the first OCT information and the second OCT information. The first OCT signal can be a calibration signal and the second OCT signal can be a sample signal. The first OCT signal can be generated as a chirped sine function sampled over a particular number of data points.

These and other objects, features and advantages of the exemplary embodiments of the present disclosure will become apparent upon reading the following detailed description of the exemplary embodiments of the present disclosure, when taken in conjunction with the appended claims.

### BRIEF DESCRIPTION OF THE DRAWINGS

Further objects, features and advantages of the present disclosure will become apparent from the following detailed description taken in conjunction with the accompanying Figures showing illustrative embodiments of the present disclosure, in which:

FIG. 1A is an exemplary flow diagram of an exemplary method implemented using an exemplary compressed-sensed swept-source optical coherence tomography system according to an exemplary embodiment of the present disclosure;

FIG. 1B is an exemplary diagram of a data flow generated using the exemplary compressed-sensed swept-source optical coherence tomography system and/or method according to an exemplary embodiment of the present disclosure;

FIG. 1C is an exemplary diagram of a fully sampled filtered reference clock signal according to an exemplary embodiment of the present disclosure;

FIG. 1D is an exemplary diagram of a randomly generated mask according to an exemplary embodiment of the present disclosure;

FIG. 1E is an exemplary diagram of a downsampled signal according to an exemplary embodiment of the present disclosure;

FIG. 1F is an exemplary diagram of a reconstructed signal according to an exemplary embodiment of the present disclosure;

FIG. 2 is an exemplary diagram of a software-based approach for the exemplary compressed-sensed swept-source optical coherence tomography system and method according to an exemplary embodiment of the present disclosure;

FIG. 3 is an exemplary combined system and flow diagram implementing a hardware-based approach for the exemplary compressed-sensed swept-source optical coherence tomography system and method according to an exemplary embodiment of the present disclosure;

FIG. 4 is an exemplary diagram of hardware-based sub-sampling according to an exemplary embodiment of the present disclosure;

FIGS. 5A-5D are exemplary graphs of exemplary calibration signals acquired in a SS-OCT system according to an exemplary embodiment of the present disclosure;

FIGS. 6A-6E are exemplary graphs of exemplary results of CS-enabled calibration according to an exemplary embodiment of the present disclosure;

FIGS. 7A-7D are graphs illustrating exemplary results using the exemplary compressed-sensed swept-source optical coherence tomography system and method according to an exemplary embodiment of the present disclosure;

FIGS. 8A and 8B are further graphs illustrating additional exemplary results using the exemplary compressed-sensed swept-source optical coherence tomography system and method according to an exemplary embodiment of the present disclosure;

FIG. 9A is an exemplary graph illustrating an exemplary quality of phase reconstruction for an instantaneous phase at the peak (e.g., mirror) location of the reference clock signal from original signal and reconstructed signal over 8,000 A-lines according to an exemplary embodiment of the present disclosure;

FIG. 9B is an exemplary graph illustrating the exemplary quality of phase reconstruction for a zoomed-in view for the first 200 A-lines according to an exemplary embodiment of the present disclosure;

FIGS. 10A-10D are ex diagrams of numerical simulations according to an exemplary embodiment of the present disclosure;

FIGS. 11A and 11B are exemplary graphs of the results of a vibrational test according to an exemplary embodiment of the present disclosure;

FIGS. 12A-12D are exemplary images of the results of a flow velocity measure according to an exemplary embodiment of the present disclosure;

FIGS. 12E and 12F are exemplary Doppler constructed images according to an exemplary embodiment of the present disclosure;

FIG. 12G is an exemplary graph of an averaged depth profile according to an exemplary embodiment of the present disclosure;

FIGS. 13A-13D are diagrams showing the mis-synchronization due to the usage of two digital acquisition boards according to an exemplary embodiment of the present disclosure;

FIG. 14A is an exemplary flow diagram of an exemplary method for compressing data that is based on an optical coherence tomography signal according to an exemplary embodiment of the present disclosure;

FIG. 14B is an exemplary flow diagram of the exemplary method for compressing the optical coherence tomography signal according to an exemplary embodiment of the present disclosure; and

FIG. 15 is an illustration of an exemplary block diagram of an exemplary system in accordance with certain exemplary embodiments of the present disclosure.

Throughout the drawings, the same reference numerals and characters, unless otherwise stated, are used to denote like features, elements, components or portions of the illustrated embodiments. Moreover, while the present disclosure will now be described in detail with reference to the figures, it is done so in connection with the illustrative embodiments and is not limited by the particular embodiments illustrated in the figures and the appended claims.

### DETAILED DESCRIPTION OF EXEMPLARY EMBODIMENTS

The problem of CS can include recovering a signal x∈ [symbol] ^{M} that can have a sparse structure, from a linear observation y=Φx∈ [symbol] ^{P}, where Φ∈ [symbol] ^{P×M} can be called the measurement operator, even when the number of observations can be drastically lower than the size of the signal to recover (e.g., P<<M). The problem y=Φx can be strongly ill-posed, while the prior information on the sparsity of the signal x can be beneficial. More generally, the signal x can have a sparse representation in a known dictionary Ψ∈ [symbol] ^{M×L}, meaning that there can exist a vector s∈ [symbol] ^{L} such that x=Ψs with very few non-zero coefficients. Ψ can be called the sparsifying transform, and s can be said S-sparse if only S of its coefficients can be non-zero the CS theory shows that.

Under some constraints (see, e.g., Reference 39), it can be possible to recover an estimator \hat{x} of the true signal x from the observation y by solving the following exemplary optimization equation:

$$\hat{x} = {argmin}_{x ∈ {ℂ}^{M}} \vert \vert {Ψ}^{*} x {\vert \vert}_{1} s . t . Φ x = y \qquad ( 1 )$$

where Ψ* can be the pseudo inverse of the operator Ψ. The quality of the estimation \hat{x} of the ground truth x can depend on several factors: both matrices Φ and Ψ, the level of sparsity of x, and the intensity of noise in the observation.

In addition, in certain cases of noiseless acquisition, the minimum number M of measurements utilized for exact reconstruction can be given by, for example:

P≥Sμ(Φ,Ψ)² log M (2)

where μ(Φ, Ψ), can measure the incoherence between the matrices Φ and Ψ, and S can be the sparsity level of x. (See, e.g., References 46 & 47).

The exemplary system, method, computer-accessible medium, and apparatus, according to an exemplary embodiment of the present disclosure, can be used to address the mismatch that exists in high speed phase-resolved optical coherence tomography. A reference clock channel can be downsampled on a hardware or software level, and the original signal can be recovered using CS. Thus, only a fraction of the reference clock data is needed and transferred during the image acquisition.

For most prior SS-OCT systems, a simultaneously recorded reference clock channel is typically needed to calibrate the wavelength-scanning curve. For phase-sensitive measurements, both the sample channel and the clock channel can be over-sampled to improve the phase stability. Given the already high data rate of SS-OCT systems (e.g., about 800 MB/s for regularly sampled sample channel), the over-sampled dual-channel configuration can impose a significant burden. However, the reference clock channel can include numerous redundant areas, and can be a spectrally sparse signal. Thus, CS can be utilized to reduce the data rate for transfer and/or storage. This can be performed according to exemplary embodiments of the present disclosure using, for example, (i) an exemplary hardware-based approach, which can include decimating the signal before digitization (e.g., reducing the data rate in transfer and storage) or (ii) an exemplary software-based approach to decimate the signal after digitization and transportation (e.g., to help reduce the storage), and/or (iii) a combined hardware and software-based approach.

The framework of CS can facilitate the reconstruction of a sparse signal x∈C^{N} from a vector of observations y=Φx∈C^{M} constituted by linear projections of x, where the number of projections can be significantly smaller than the size of the signal (M×N). (See, e.g., References 8 and 9). In the case of SS-OCT, the acquisition system of the reference clock data and the natural redundancy of this kind of signal can facilitate a reduction in the data size using a set of subsampled clock signal data, an almost exact estimate of the true signal can be recovered, using, for example, the properties of convex optimization in the Fourier domain.

### Exemplary Method

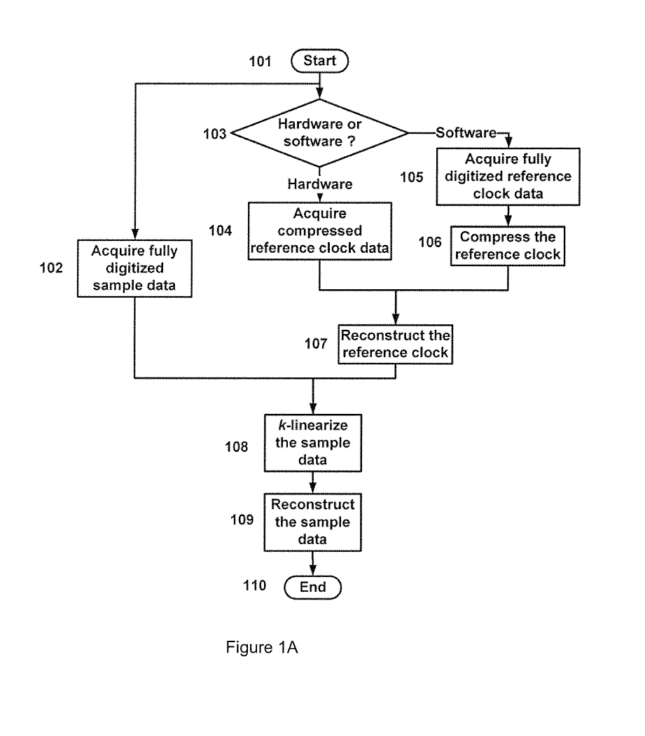

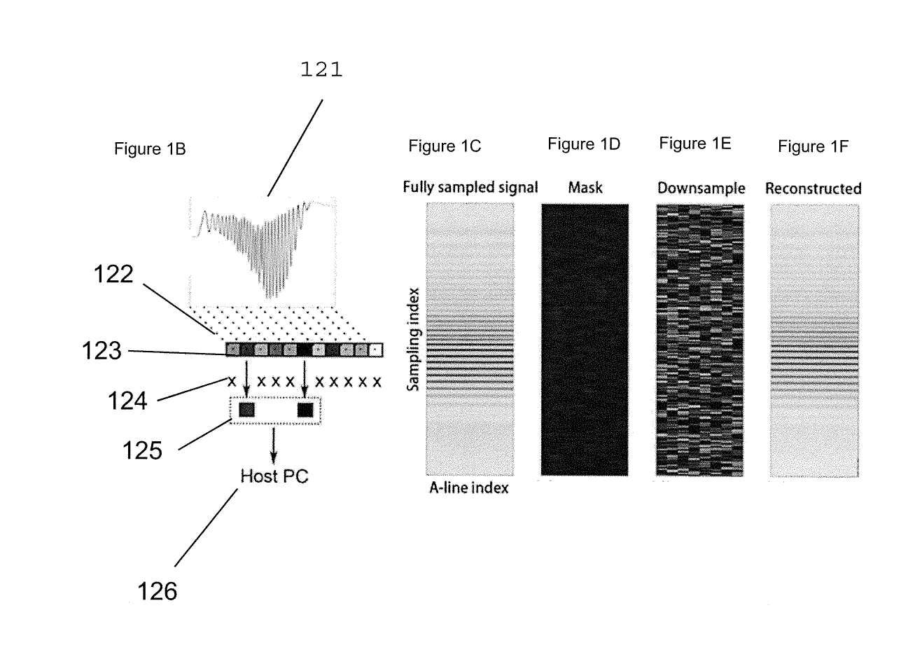

FIG. 1A shows an exemplary flow diagram of an exemplary method implemented with an exemplary CS-SS-OCT system according to an exemplary embodiment of the present disclosure. For example, at procedure 101, the exemplary method can begin. At procedure 102, the sample (e.g., image) channel can be fully digitized by the data acquisition (“DAQ”) board. At procedure 103, a determination can be made as to whether to implement a hardware enabled/facilitated version of CS, a software enabled version of CS, or a combination of the two. If the exemplary hardware version is enabled/facilitated, then at procedure 104, the down-sampled reference clock can be acquired and transferred to the host computer. If the exemplary system is software enabled/facilitated, then at procedure 105, the fully digitized reference clock and the data can be transferred to the host computer. At procedure 106, the down-sampling process can be performed on the host memory. At procedure 107, the down-sampled reference clock can be reconstructed using CS. At procedure 108, a wavelength sweeping curve can be extracted from the reference cock and can be used to resample the sample data. At procedure 109, the image can be reconstructed based on k-linearized sample data. At procedure 110, the exemplary method can end.

FIG. 1B shows an exemplary diagram of the data flow of the exemplary CS-enabled SS-OCT system and method according to an exemplary embodiment of the present disclosure. For example, an analog signal can be input at procedure 121 and digitally converted at procedure 122. At procedure 124, a binary mask with a compression ratio (“CR”) of 4 (see e.g., diagram shown in FIG. 1D) can be randomly generated and repeatedly applied on the fully digitized data stream 123 as shown in the diagram of FIG. 1C. The data can be down sampled at procedure 125. Only a fraction of the data selected by the binary mask can then be transferred to, and stored in, the host 126 PC (e.g., in RAM, ROM, etc.). The down-sampled signal and reconstructed results are displayed in diagrams shown in FIGS. 1E and 1F, respectively. The exemplary masking procedure can be implemented in Matlab on a PC workstation (2.93 GHz Quad-core CPU with 8 GB of RAM). For example, the execution time can be about 16 seconds for a complete 2048×1600 clock signal. The masking can also be implemented directly onboard (e.g., on the DAQ) for faster computation and easier use.

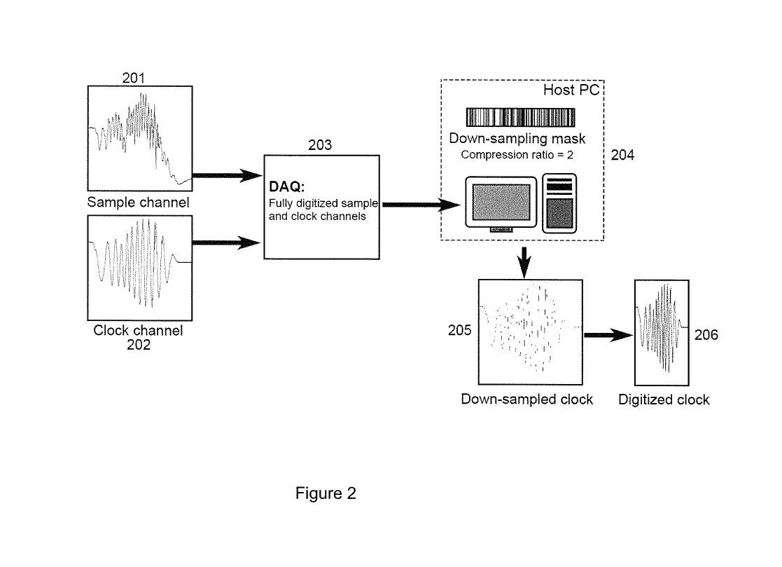

FIG. 2 shows an exemplary diagram of a software-based approach for the exemplary CS-enabled SS-OCT system and method according to an exemplary embodiment of the present disclosure. For example, Block 201 is an analog sample channel, and Block 202 is an analog clock channel. DAQ board 203 can receive the sample channel 201 and the clock channel 202. DAQ board 203 can be used to digitize both channels at a full rate. The digitized data can be transferred to the host computer 204, which can be used to generate a down-sampling mask based on particular predefined rules. Host computer 204 can be used to decimate (e.g., down sample) the fully sampled clock data. Block 205 is an exemplary down-sampled clock, where the discontinuous data points represent those that have been deleted. Block 206 is an exemplary output stream of down-sampled clock data.

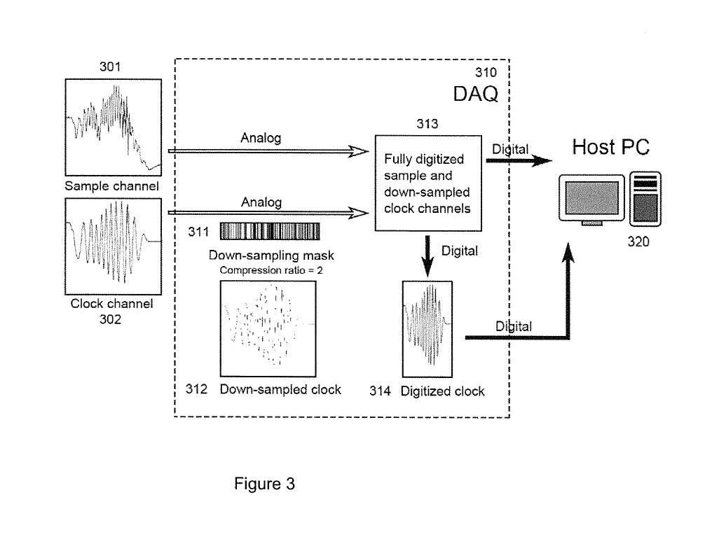

FIG. 3 illustrates an exemplary combined system and flow diagram implementing a hardware-based approach for the exemplary CS-enabled SS-OCT tomography system according to an exemplary embodiment of the present disclosure. For example, Block 301 is an exemplary analog sample channel and Block 302 is an exemplary analog clock channel. DAQ board 310 can receive the full signal of the sample channel 301 and a down-sampling mask 311 can be used to down sample the clock channel 302, which can be generated based a certain rules or procedures and implemented using exemplary hardware, prior to being received by DAQ board 310. The digitization procedure can follow both the sampling clock and the mask: only the sampling point that is not masked out can be digitized and acquired by the DAQ board. Block 312 is an exemplary down-sampled clock, where the discontinuous data points can represent those that have been deleted. At procedure 313, the DAQ board can digitize the sample channel at the full rate and the clock channel at a reduced rate. Block 314 is an exemplary output stream of down-sampled clock data. Host PC 320 can be provided with the fully digitized sample data and the compressed clock data.

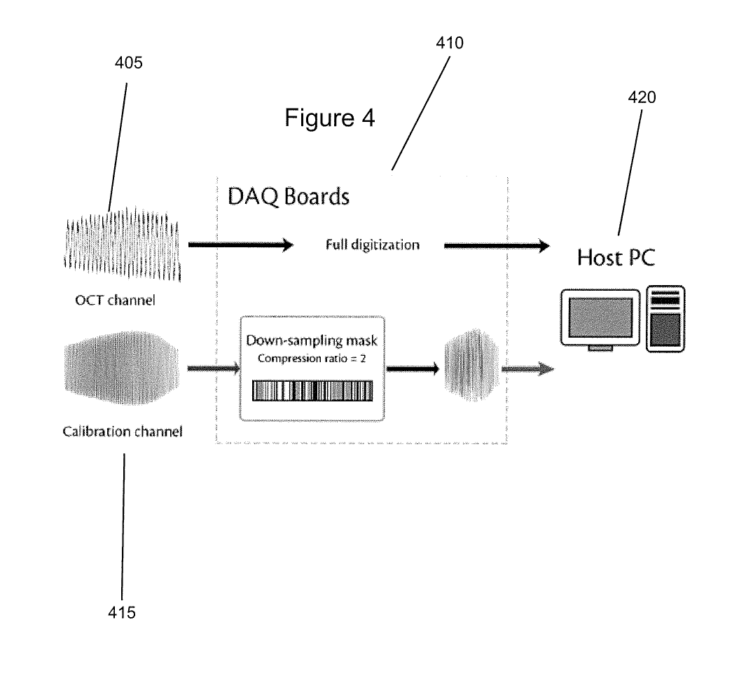

FIG. 4 shows a further exemplary schematic diagram of the exemplary hardware-based signal sub-sampling according to an exemplary embodiment of the present disclosure. The OCT channel 405 can be digitized by a DAQ board 410 (e.g., ATS 9373, AlazarTech, Canada) at full speed, while the simultaneously recorded calibration channel 415 can be digitized by the same or a different DAQ board 410, which can have the same specifications, or can have a reduced rate via random sub-sampling. Specifically, a set of 65,536 registers, which can be referred as “skip bitmap”, can be used to specify which trigger event is to be ignored during the acquisition by assigning binary values to the corresponding registers. Whenever a trigger event can be skipped, no data can be generated. The skip bitmap can be made any arbitrary pattern and manipulated via the provided software API (e.g., ATS-SDK V7.1.4 (C++), AlazarTech, Canada).

For example, a vector of 4,000 bits can be loaded, which can include alternating 1s and 0s, onto the skipping table. The DAQ board 410 can digitize the signal when every other trigger can be received during the first 4,000 clock periods. This process can be repeated for the next 4,000 sampling points and so forth. Thus, a sub-sampled calibration signal can be generated whose down-sampling factor can be 2.

The fully sampled OCT signal and the sub-sampled calibration signal can be later transferred to the host computer 420 via a Peripheral Component Interconnect Express (“PCI-e”) Gen. 3×8 bus. The two exemplary DAQ boards have to be carefully connected and synchronized to avoid extra jitter.

To implement the exemplary apparatus, a custom SS-OCT was prepared. A DAQ (e.g., Alarartech, ATS 9373, USA) was configured to record a down-sampled version of the reference clock. After obtaining the down-sampled data, the reconstruction was performed using a fast method for sparse recovery (e.g., a NESTA procedure).

The exemplary SS-OCT system included was (i) a MEMS-based swept source (e.g., HSL-20-100-M-3-S, Santee, Japan), and (ii) an integrated Mach Zehnder interferometer (e.g., INT-MZI-1300, Thorlabs, USA).

The center wavelength of the system was 1317.5 nm, the axial resolution was 16.8 μm, and the lateral resolution was 8.77 μm. The 6 dB coherence length was 6.5 mm, and the SNR of the system was 105.4 dB.

### Exemplary Direct Domain Compressed Sensing

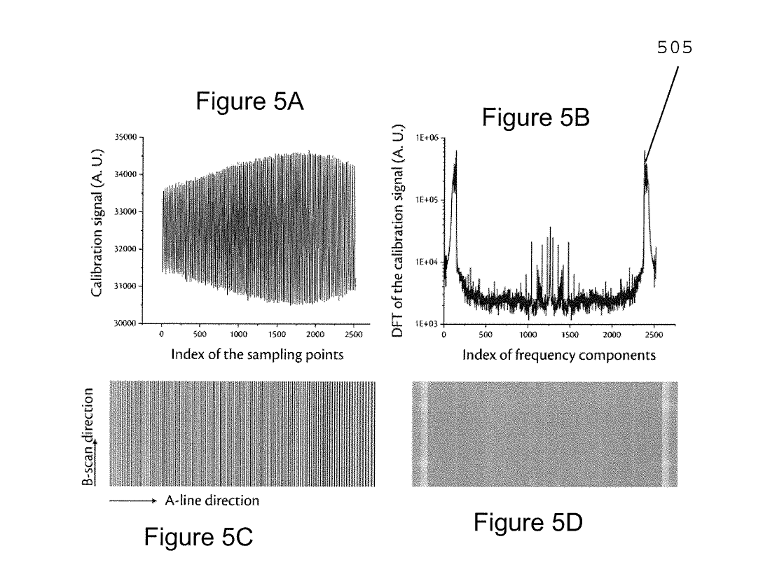

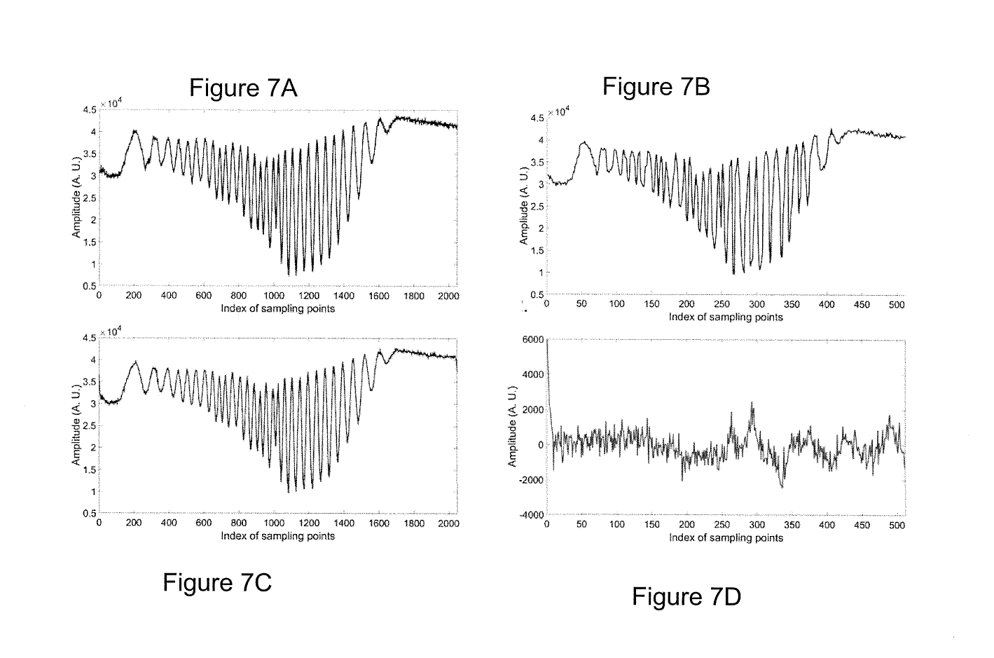

FIGS. 5A-5D show exemplary calibration signals acquired in an exemplary SS-OCT. FIG. 5A shows 1D visualization of one calibration A-line. FIG. 5B shows 1D Fourier Transform of FIG. 7A (e.g., logarithmic view). FIG. 5C shows 2D visualization of a calibration B-scan. FIG. 5D shows 2D Fourier Transform of FIG. 5C (e.g., logarithmic view).

The calibration signal in SS-OCT can have two or more properties that can be used for the exemplary CS-based procedure, suited for faster SS-OCT acquisition:

First, the single calibration signal, corresponding to a single A-line 505, can be a chirped sinusoidal function. (See, e.g., Reference 33). Such signal can have a highly localized spectrum. These signals have sparse Fourier transform (see e.g., FIG. 5B), which can be the best case scenario for direct domain CS acquisition, and such signal can be reconstructed with a high precision from very few randomly acquired samples.

Second, any consecutive calibration signals (e.g., corresponding to consecutive A-lines, during the raster scanning of the whole sample) can have a very similar appearance. Up to a few exceptions, where notable pixel shifts can be observed, the pixel-wise difference of two consecutive calibration signals can be equal to a constant. Thus, if the whole stack of calibration signals can be considered to be a calibration B-scan, the resulting image can present a strong redundancy along the fast scanning axis (see e.g., FIG. 5C). This redundancy can be exploited with CS reconstruction results improved when considering calibration signals as two dimensional images.

Most of state-of-the-art CS applications (e.g., such as MRI (see, e.g., Reference 48), Astrophysics (see, e.g., Reference 49), Radar (see, e.g., Reference 50), Holography (see, e.g., Reference 51) can exploit the natural sparsity of signals in the direct domain (e.g., W being equal to the Identity matrix, or to a wavelet transform) or of their gradient (e.g., W being the Total Variation operator), while sampling the data in an incoherent space, such as the Fourier or the Fresnel domain. The exemplary system, method, and computer-accessible medium, according to an exemplary embodiment of the present disclosure, can be performed in the direct domain, and the sparsifying transform can correspond to the Fourier transform. Φ∈{0,1}^{P,M} can be defined as a random selection matrix, such that, for a calibration signal x, the resultant signal y=Φx can be a random sub-sampling of the measures of x. The matrix Φ can contain at most 1 non-zero coefficient on each line, for a total amount of P non-zero coefficients, and y can be a selection of P samples among the M that define x. In addition, Ψ= [symbol] ⁻¹ can be defined, which can represent the inverse Fourier transform operator. Then, an estimator of x can be recovered, with high precision, from the acquisition y by solving, for example:

$$\hat{x} = {argmin}_{x ∈ {ℂ}^{M}} \vert \vert ℱ ( x ) {\vert \vert}_{1} s . t . Φ x = y . \qquad ( 3 )$$

When x represents a group of Nₖ clock signals instead of only one clock signal, the approach can be exactly the same, where the operator [symbol] can denote the 2D-Fourier transform instead of the 1D-Fourier transform, and the selection matrix Φ can belong to {0,1}^{N}^{ₖ}^{P×N}^{ₖ}^{M}.

### Exemplary 1D VS. 2D

Equation (3) provides an estimator of a calibration signal x from only a fraction of its samples, denoted as y. The exemplary system, method, and computer-accessible medium, according to an exemplary embodiment of the present disclosure, can utilize two different exemplary approaches for the acquisition of y. While the mathematical formulation can be the same, the implementations can be different.

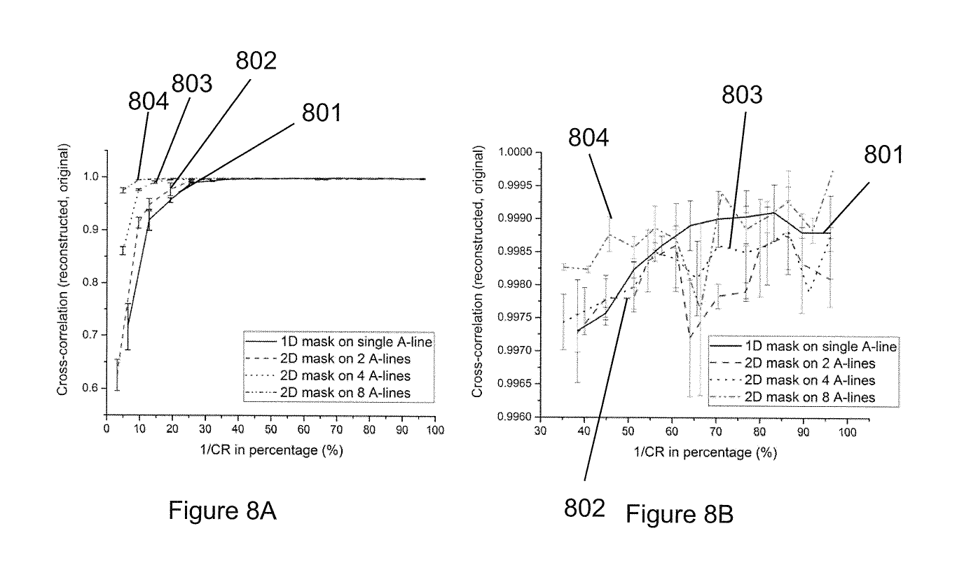

One calibration A-line can be partially acquired and reconstructed as an estimation through CS optimization. This can be one-dimensional, can exploit the sparsity of the Fourier transform of each of the clock signals (see e.g., FIG. 8B), and can be solved using a multitude of exemplary procedures. (See, e.g., References 52 and 53). Then, the procedure can be run on the N calibration A-lines, for a complete reconstruction of the calibration B-scan.

The acquired data y can represent the sub-sampling of a group of Nₖ calibration A-lines, where Nₖ can be an integer between 2 and 8 with the exemplary set-up. The exemplary system, method, and computer-accessible medium, according to an exemplary embodiment of the present disclosure, can utilize the sparsity of the 2D Fourier Transform of the M×Nₖ portion of the calibration B-scan. Then, the procedure can be run on the N/Nₖ groups of calibration A-lines, leading to the reconstruction of the complete calibration B-scan.

### Exemplary Optimal Sampling Rate for CS-SS-OCT

Using various notations (see, e.g., Reference 31), the clock signal can be modeled as a chirped sine function that can be sampled regularly over M data points. Thus, for example:

I[m]=A cos(2k[m]z_{d}). (4)

where A can be the emission spectrum function, supposed to be constant, k can be the output wavenumber which can be a function of time (e.g., sample number in discrete domain), and z_{d} can be the optical path length difference between both mirrors, which can be fixed for calibration channel.

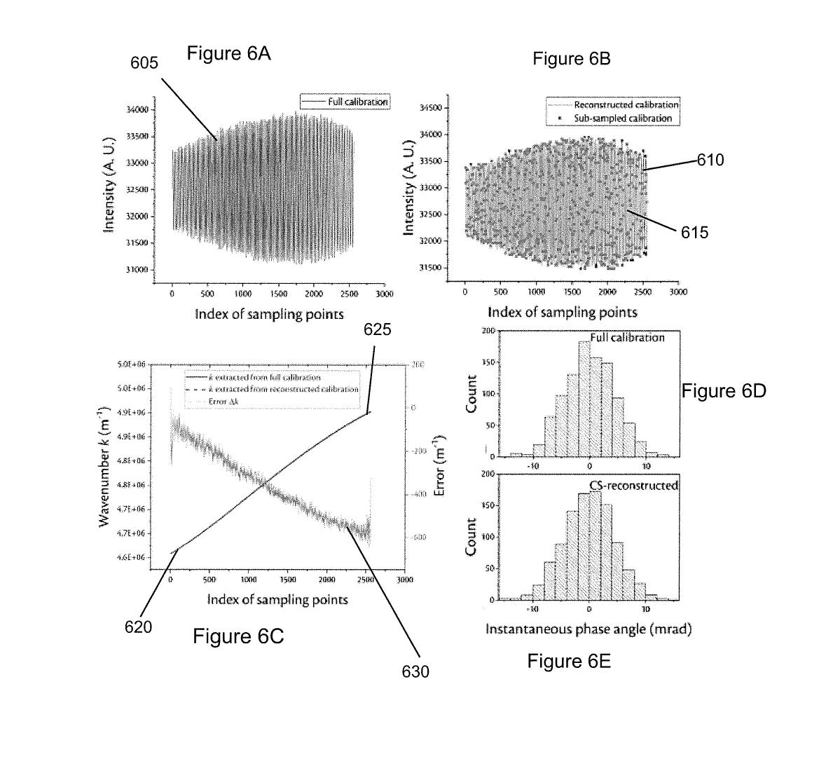

FIGS. 6A-6E show the exemplary CS-enabled calibration method utilizes less data bandwidth without compromising phase stability. FIG. 6A shows the Full calibration signal 605. FIG. 6B shows the experimentally obtained sub-sampled calibration signal 610 and its reconstruction calibration signal 615. FIG. 6C shows the k-t curves extracted from the full calibration signal 620 and the reconstructed signal 625. The error 630, whose amplitude was less than 0.1% of that of the original signal over the entire spectrum, was also plotted. The histogram of the instantaneous phase angles obtained from the peak location of OCT images by using full calibration signal and reconstructed signal are shown in FIGS. 6D and 6E. The standard deviation of the distribution was 4.53 mrad and 4.49 mrad, respectively.

For example, the k-t curve can be linear, leading to the calibration signal I being a perfect sinusoidal function. However, the actual function k of the laser can be nonlinear. (See e.g., FIG. 6C). The consequence of the nonlinear k-t curve can be that the calibration signal can consist in a sine wave with modulated amplitude, to which sine waves of lower frequency can be added. If the Fourier transform of the calibration signal is utilized, two peaks corresponding to the highest frequency contained in the signal, of rather thin widths (e.g., less than 100 coefficients overall), can be observed. (See e.g., FIGS. 5A-5D). Considering the other Fourier coefficients as noise terms, it can be determined that the sparsity S of the clock signal can be given by the number of coefficients contained at these two locations. Thus, since the mutual coherence μ(Φ, Ψ) can be close to 1 in the case of direct-domain compressed sensing, the sub-sampling rate utilized for almost exact reconstruction of the clock signal can be given by, for example:

$$\frac{P}{M} ∼ {S}^{′} \frac{log M}{M} \qquad ( 5 )$$

where S′ can be an unknown constant, close to S.

### Exemplary CS Signal Reconstruction

In order to reconstruct the calibration signal from the sub-sampled data, the CS problem can be solved using an exemplary NESTA procedure. (See, e.g., Reference 54). The exemplary procedure was implemented using Matlab, on a PC workstation 2.93 GHz quad-core CPU, with 8 GB of RAM.

The l₁ version of the NESTA procedure, adapted to the operators Φ and Ψ defined above was used. In addition, the regularization parameter ε can be set to 0 in the exemplary experiments, in order to achieve an exact, or near exact, reconstruction.

With these conditions, the reconstruction time of one calibration A-line can be, for example, between about 10 and about 50 ms, depending on the sampling rate, and the sampling strategy.

### Exemplary Reconstruction Quality: Numerical Simulation

The impact on reconstruction quality of different sub-sampling mask configurations including sub-sampling rate (P/M) and mask width (Nₖ) were examined. Conventional SS-OCT calibration data were obtained at 200 Mega Samples/s (MS/s) and 1.6 GS/s for the numerical simulation. Random sub-sampling bitmaps were computer-generated and were used to digitally mask the calibration signal. The sub-sampled data was then reconstructed using the exemplary NESTA procedure. To evaluate the reconstruction quality, the correlation coefficient r_{x,\hat{x}} between the original signal x and the reconstructed signal \hat{x}, was used, which can be given by, for example:

$${r}_{x , \hat{x}} = \frac{{Σ}_{i = 1}^{n} ( {x}_{i} - \overline{x} ) ( {\hat{x}}_{i} - \overline{x} )}{\sqrt{{{Σ}_{i = 1}^{n} ( {x}_{i} - \overline{x} )}^{2} \sqrt{{{Σ}_{i = 1}^{n} ( {\hat{x}}_{i} - \overline{x} )}^{2}}}} \qquad ( 6 )$$

where Λ r_{x,\hat{x}} close to 1 can indicate a good reconstruction.

### Exemplary Phase Stability

The exemplary system, method, and computer-accessible medium, according to an exemplary embodiment of the present disclosure was evaluated using the reconstructed calibration signal to remap and stabilize the corresponding OCT spectrum. A “standardized test” was conducted to measure phase stability of the exemplary system as described below.

A microscope slide (e.g., 1 mm thick, Microscope Slides, Fisherfinest, USA) was placed under the sample arm, and blocked the reference arm. The interference pattern between the light reflected from the top surface and the bottom surface of the sample was obtained from the DAQ board #1 at 800 MS/s. The sub-sampled calibration signal was recorded by DAQ board #2 according to a predefined mask (P/M=0.3, Nₖ=1), while the same calibration signal was fully digitized by DAQ board #1 for comparison purposes. Approximately 1,000 M-scans were obtained at a fixed sample location.

The calibration signal was reconstructed from its sub-sampled copy, and used this reconstructed signal was used to remap the OCT signal. The instantaneous phase angles over time at the peak location of the OCT A-lines were extracted and their standard deviation was calculated.

The exemplary system configuration can be identical to that reported above. SDPM was used to measure the vibrational frequency of the sub-sampling-interval displacements of a sample to verify the exemplary system's capability of conducting sensitive phase measurements. The test arrangement included a piezoelectric actuator (e.g., PZS001, Thorlabs, USA), which was driven by a sinusoidal AC voltage from a function generator (e.g., AFG3022C, Tektronix, USA). The frequency of the driving sinusoidal was about 10 kHz and the peak-to-peak voltage was about 1V.

To illustrate the performance of the exemplary system, two other remapping procedures (see, e.g., References 32 and 34 were used on the same OCT dataset, for comparison.

### Exemplary Doppler OCT

To further validate the exemplary system and method, an experimental phantom was constructed to mimic blood flow. Intralipid emulsion (e.g., Sigma Aldrich, USA) was diluted to a concentration of about 0.25% in de-ionized water and stored within an intravenous fluid bag and tubing setup. The tubing was fitted into an irrigation pump (e.g., CoolFlow, Biosense Webster, USA) which facilitated precise manipulation of laminar flow rates. Imaging was performed over the short axis of the tubing for flow rates ranging between about 1-3 mL/min.

### Exemplary Results

The exemplary system, method, computer-accessible medium, and apparatus according to an exemplary embodiment of the present disclosure can include a phase resolved SS-OCT, which can be used to experimentally evaluate the phase reconstruction quality in addition to the intensity reconstruction quality. For example, two identical DAQ boards were installed: one—configured in down-sampling mode, while the other—recorded the fully sampled signal. Both boards were synchronized to the same triggering signal to minimize the timing jitter.

FIGS. 7A-7D show graphs illustrating exemplary results using the exemplary CS-enabled SS-OCT system and method according to an exemplary embodiment of the present disclosure. Such exemplary graphs include an illustration of (i) an exemplary reference clock A-line (e.g., graph shown in FIG. 7A), (ii) a fully sampled A-line with 2048 sampling points (e.g., graph shown in FIG. 7B), (iii) an A-line down-sampled 4 times, for example, 512 sampling points (e.g., graph shown in FIG. 7C), and (iv) a reconstructed A-line with 2048 sampling points (e.g., graph shown in FIG. 7D) and the error rate between graphs 701 and 703.

For an exemplary intensity reconstruction, the cross-correlation was calculated between the reconstructed signal and the fully sampled signal. The size of the exemplary mask and the exemplary compression ratio were varied, and the mean cross-correlation was calculated over 1000 A-lines. The exemplary results are provided in the graphs shown in FIGS. 8A and 8B. As shown in FIGS. 8A and 8B, a larger mask can provide a better reconstruction result when the CR can be high (e.g., greater than 3). The reconstruction precision for a different mask width is shown in the graph of FIG. 8A. The cross-correlation between the original signal and the reconstructed signal was determined by applying a single A-line (e.g., line 801), 2 A-lines (e.g., line 802), 4 A-lines (e.g., line 803), and 8 A-lines mask (e.g., line 804). A wider mask shows reconstruction quality at higher CR (e.g., graph shown in FIG. 8B) with a magnified view of graph 801 in the lower CR region.

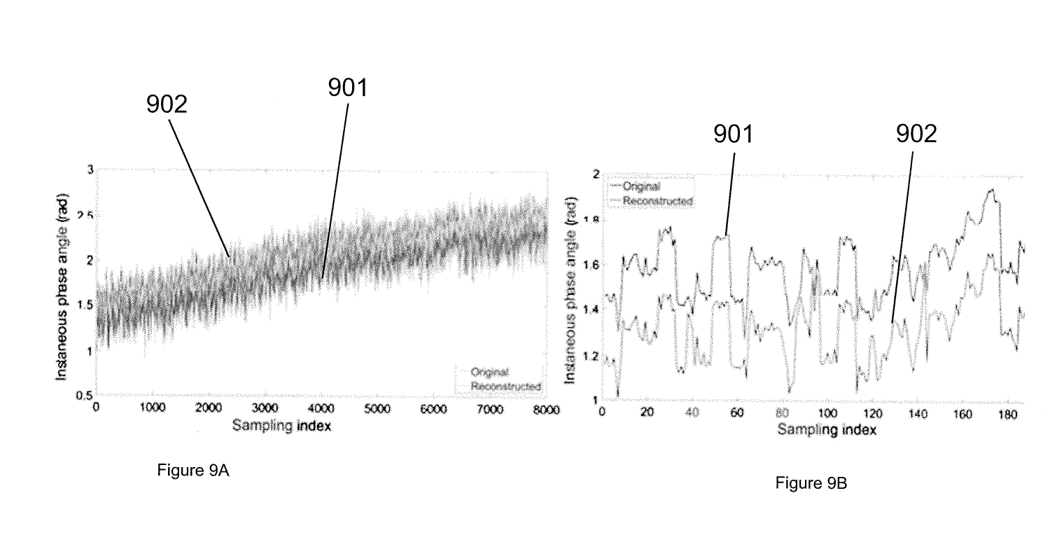

The phase of the reconstructed signal was evaluated; the instantaneous phase at the peak location of both fully-sampled signal (e.g., signal 901) and the reconstructed signal (e.g., signal 902) are plotted in FIG. 6A. The extracted phase of the reconstructed signal 902 follows that of the original, fully-sampled, signal 901, except for a fixed offset due to the slight jitter between the DAQ boards. The mean square root error can be, for example, 6.57 mrad after correcting for the jitter. For example, the measured phase noise of the original, fully-sampled, signal 901 can already be 46.19 mrad, which suggests that the reconstruction error can be below the phase noise presented in the clock channel A zoomed-in view is provided in FIG. 9B for better visualization. The reconstructed phase generally follows the trends of the original signals with couple exceptions.

### Exemplary Reconstruction Quality Evaluation

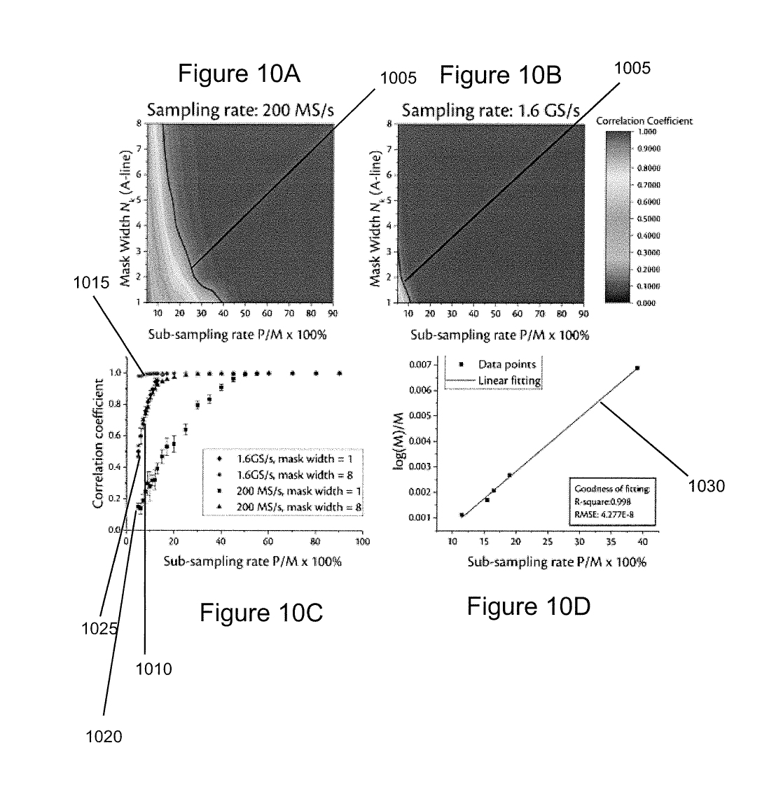

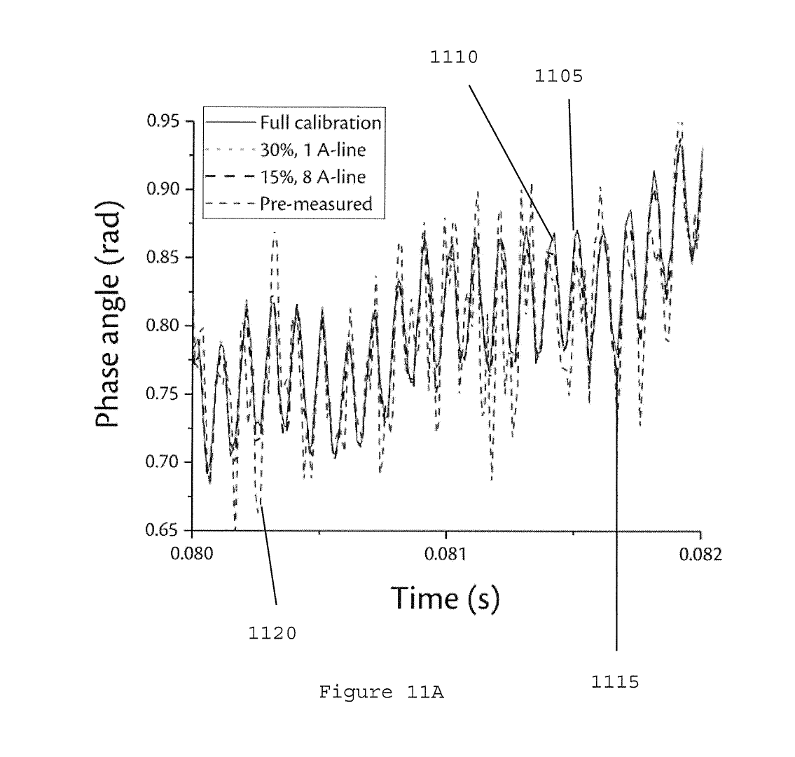

FIGS. 10A-10D shows Numerical Simulation Correlation coefficient r̅_{x,\hat{x}} between the reconstructed signal \hat{x} and the original signal x at 200 MS/s (see e.g., FIG. 10A) and 1.6 GS/s (See e.g., FIG. 10B). The solid curves 1005 in FIGS. 10A and 10B provide the levels that correspond to r̅_{x,\hat{x}}=0.9. FIG. 10C shows the evolution of r̅_{x,\hat{x}} against sub-sampling rate P/M for different mask width Nₖ (e.g., element 1010 shows 1.6 GS/s with a mask width of 1, element 915 shows 1.6 GS/s with a mask width of 8, element 1020 shows 200 MS/s with a mask width of 1, and element 1025 shows 200 MS/s with a mask width of 8). FIG. 10D shows that the utilized sub-sampling rate for good reconstruction (e.g., to achieve r̅_{x,\hat{x}}=0.9) fits linearly against log M/M (as shown by line 1030), which agrees with the exemplary prediction made in Equation 5. The sampling rate of the DAQ board was first set as 200 MS/s so that each A-line contained about 1,000 sampling points (e.g., M=1, 000). FIG. 10A show pseudo-color maps of r̅_{x,\hat{x}} under different experimental conditions. Taking into account the randomness of the masks, five simulations were conducted for each sampling rate, and the average r̅_{x,\hat{x}} over these five simulations was computed.

As shown in FIG. 10C, solid curve 1020 can be the contour line that can be given by r̅_{x,\hat{x}}=0.9, which can be used as a baseline. It shows that for a mask that consists of only one A-line, a sub-sampling rate (P/M)>40% can be beneficial to achieve a result better than the baseline. Generally, r̅_{x,\hat{x}} can decrease exponentially with P/M. P/M can be reduced to approximately 15% if mask width Nₖ can be set to 8, due to the redundancy within B-scans.

The exemplary experiment was repeated for a different sampling rate (e.g., 1.6 GS/s), and the results are shown in FIG. 10B. The same reconstruction quality r̅_{x,\hat{x}} can be achieved at a lower sub-sampling rate/larger mask width: For example, by sub-sampling only 5% of the data (Nₖ=8), the averaged correlation coefficient between the original calibration signal and the reconstructed one could be as high as 0.98. To illustrate this, r̅_{x,\hat{x}} was plotted against the sub-sampling percentage for four combinations of sampling rates and mask widths in FIG. 10C.

Additional experiments were conducted for three other sampling rates: 600 MS/s, 800 MS/s and 1 GS/s. The P/M (Nₖ=1) that can be needed to bring r̅_{x,\hat{x}} to the baseline (e.g., 0.9) value at these sampling rates was recorded. Based on the prediction made in Equation (5), sub-sampling rates can be linear with log M/M, which fully agrees with the experimental results as shown in FIG. 9D.

### Exemplary Phase Stability

An exemplary full calibration A-line is plotted in FIG. 6A, while the sub-sampled acquisition and reconstruction are shown in FIG. 6B. The reconstructed calibration signal can be almost identical to the fully sampled one in spite of a scaling factor, which can be caused by the difference between the two DAQ boards.

The extracted k-t curve from the full and the reconstructed signals are illustrated side-by-side in FIG. 6C. The error, which can be four orders of magnitude smaller than the original signal, is shown by line 630 shown in FIG. 6C.

Additionally, a histogram of the phase angle distribution at the peak location was determined. Results, using the full and the reconstructed calibration signal, are shown in FIGS. 4D and 4E, respectively. The standard deviation of the measured phase angles can be equal to 4.53 mrad (SNR=52.65 db) for full calibration and 4.49 mrad (SNR=50.86 db) for reconstructed calibration. The phase stability of the exemplary system may not be compromised, while only 30% of the bandwidth for the extra calibration channel can be used in this example.

### Exemplary Vibrational Frequency Test

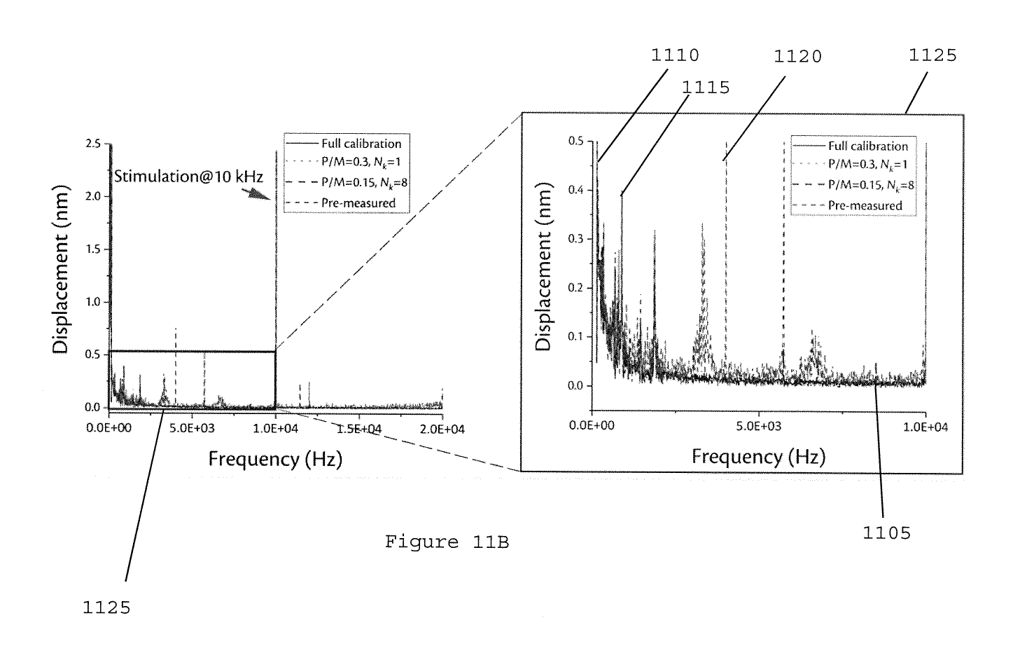

FIGS. 11A and 11B show exemplary results of the vibrational test for the Full Calibration 1105, 30%, 1 A-line 1110, 15%, 8 A-line 1115 and pre-measured signal 1120. FIG. 11A shows the instantaneous phase evolution over 2 ms and FIG. 11B shows the vibrational spectra obtained by the exemplary system along with the controls. The low frequency region (e.g., box 1125) shown in FIG. 11B can be magnified and replotted in the inset. The results of using pre-measured calibration (e.g., pre-measured signal 1120) show excessive phase noise in both time domain and spectral domain, while the others present comparable performances to that of using the full calibration. A lower sub-sampling rate (P/M) can be achieved by using a larger mask width (Nₖ) without compromising the performance. The measured noise floor for the exemplary system can be below 10 pm.

Instantaneous phase angles extracted from the surface of the piezoelectric actuator over a small time frame are shown in FIG. 11A, which shows the four different remapping strategies: pre-measured calibration 1120, full calibration 1105, CS-1 1110 (P/M=0.3, Nₖ=1) and CS-2 1120 (P/M=0.15, Nₖ=8)). Both CS results can be very similar to that of the full calibration case, while the pre-calibration result manifests apparent noises. Discrete Fourier transform was performed on the acquired phase angles, and obtained the frequency response of the actuator which is illustrated in FIG. 11B. The dominant frequency peak at 10 kHz matches that of the stimulation exactly, and can be well preserved across all methods. However, a closer look at the low frequency region of the spectra as shown in box 1125 the inset shows the elevated background noise in the case where no simultaneous calibration can be used (e.g., pre-measured 1120). Both reduced-data-rate procedures perform very well and can be comparable to that of the conventional dual-channel procedure. The noise floor of the exemplary system, method, and computer-accessible medium can be below 10 pm, if the discrete spikes that can be caused by the mechanical motion of the imaging platform can be excluded.

### Exemplary Doppler OCT

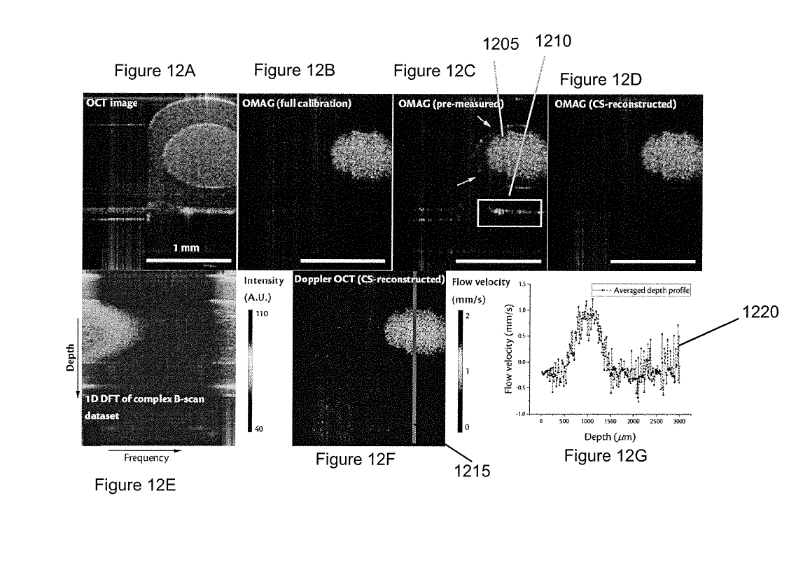

FIGS. 12A-12G show exemplary results of the flow velocity measure. FIG. 12A shows the original OCT image of the flow phantom. Its 1D DFT in fast axis direction is shown in FIG. 12E, where the Doppler frequency shift due to the presence of the intralipid flow can be observed. The resultant OMAG images by using full calibration, pre-measured calibration, and the exemplary CS-based remapping procedures are shown in FIGS. 12B, 12C and 12D, respectively. In FIG. 12C, the artifacts due to the timing jitter are shown by elements 1205 and box 1210. FIG. 12F shows the computed Doppler image by using the exemplary CS-based remapping. The averaged (e.g., 4 adjacent A-lines) depth profile 1220 at the line 1215 from FIG. 12F is shown in FIG. 12G.

The flow velocity of the irrigation pump was set to be 2 mL/min. The Tygon tubing (e.g., Saint-Gobain, France) used in the exemplary experiment had an inner diameter of about 0.89 mm and an outer diameter of about 1.56 mm. It should be noted that the tube was positioned at an angle of 86.05° to the vertical and the cross-sectional area of the tube can be 87.11% smaller than that of the pump. The original OCT image of the phantom is shown in FIG. 12A, and its DFT spectrum against the fast axis is shown in FIG. 12E. The DFT spectrum shows a strong Doppler frequency shift in the inside region of the tube. After high-pass filtering the image, the optical micro-angiography (“OMAG”) image was obtained and utilized. (See, e.g., Reference 56).

Three different remapping procedures (e.g., full calibration, pre-measured calibration, and the exemplary CS-based calibration) were used and the results are shown FIGS. 12B, 12C, and 12D, respectively. The CS-reconstructed results (P/M=0.3, Nₖ=1) shows good image quality comparable to using the full calibration. On the contrary, pre-measured calibration presents multiple artifacts due to the phase noise.

The Doppler images were computed by using the exemplary CS based method, which is shown in FIG. 12F. To further showcase the velocity profile, the average of 4 adjacent Doppler A-lines as indicated by the line 1215 in FIG. 12F was calculated and plotted in FIG. 12G. The average measured flow speed (e.g., half of the maximum velocity) was about 0.81 mm/s, which agrees with the prediction.

Both simulations and experiments conclude that the exemplary CS-based system can be capable of conducting phase-sensitive SS-OCT measures with reduced sampling data rate. Thus, phase stability of the system can be comparable to that of using full calibration

### Exemplary Hardware-Based Framework Suitable for Future Compression on Oct Channel

Although the exemplary system, method, and computer-accessible medium was only demonstrated on the calibration channel in this manuscript, the exemplary hardware configuration can be applied on the OCT channel to randomly decimate the signal. An exemplary CS-based procedure, which utilizes 1D sparsity of the A-lines, can be implemented either in real-time (see, e.g., Reference 57) or post-processing (see, e.g., Reference 42) to reconstruct the OCT images at a reduced data rate.

### Exemplary Nyquist Sampling Versus Compressed Sensing

The calibration signal shown in FIG. 8A can be band-limited, and the original signal can be reconstructed by just sampling at the Nyquist rate. However, the over-sampling of the calibration signal can be performed for the sake of better phase performance (see, e.g., Reference 31). The large uncertainty of locating the true starting time of the sweeping wavelength if using a reduced sampling rate can introduce costly timing jitters, which can compromise the phase performance of the exemplary system.

Furthermore, a much lower sampling rate than the Nyquist sampling rate can be reached with the exemplary method if the 2D redundancy of the calibration signal is exploited, as shown in FIGS. 10A-10D.

### Exemplary Optimal Sampling Rate

The minimum sampling rate facilitated for exact reconstruction of a clock signal can be linear with log M/M, with a constant that can be close from the sparsity level of the signal according to Equation 5.

### Exemplary Real-Time Processing

Based on the independent nature of the OCT dataset, it can be possible to introduce parallel computing to accelerate the processing. (See, e.g., Reference 57). A graphics processing unit (“GPU”) based package can be implemented to facilitate real-time reconstruction and OCT image visualization.

### Exemplary Synchronization Between the DAQ Boards

One of the challenges was to synchronize the two DAQ boards in the experiment, so that both the sub-sampled signal and the fully digitized control signal can be perfectly aligned. A single DAQ board may not be used to simultaneously perform the full digitization of OCT channel and the sub-sampling of the calibration channel because certain DAQ boards may only support one mode at a time.

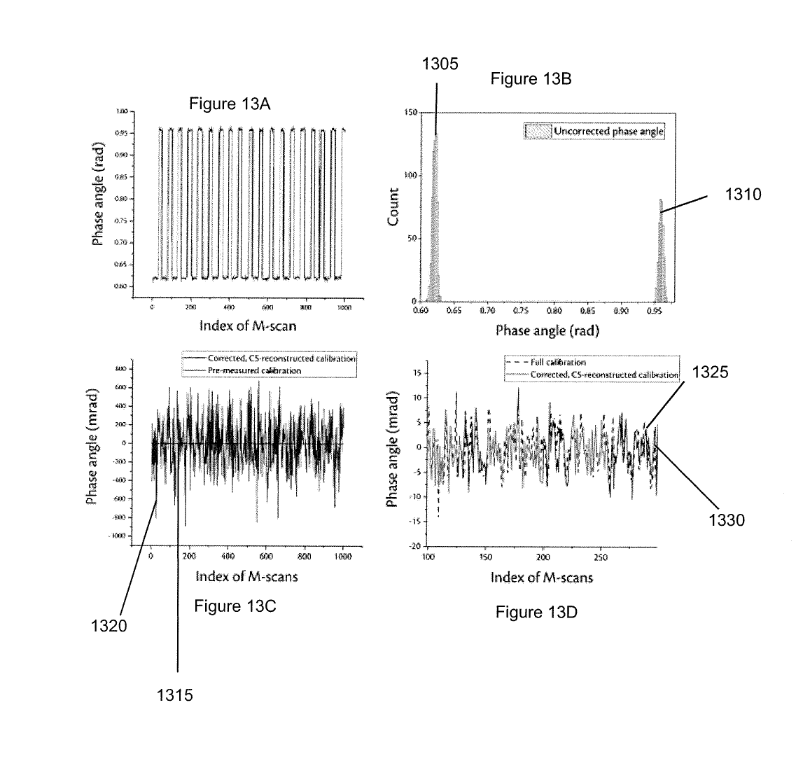

In the exemplary setup, the two boards can be ensured to be of the exact same specifications and can be connected to the same trigger signal through same length of electrical connections. But there can be still a random jitter that can be smaller than 1 clock period between the two boards due to the random starting phase of their crystal oscillators. In fact, if the OCT signals can be remapped by using reconstructed calibration signals as described above, a fixed amount of jump in the phase can be observed as shown in FIG. 13A. The histogram of the uncorrected phase angles, which is shown in FIG. 13B, shows two peaks and two groups of distributions 1305, 1310. One group corresponds to the reconstructed calibration that can have no jitter, and one corresponds to the missed one clock period.

This jitter would not exist if a device can be used which can support both modes at the same time. For example, a dual channel DAQ board with a customized field-programmable gate array (“FPGA”). Therefore, the numerical correction was performed by measuring the value between the two peaks and subtracting it from the group that can have the high value. The corrected results 1315 are shown in FIG. 13C along with the results using pre-measured calibration 1320. A side-by-side comparison of the full calibration 1325 and the corrected results 1330 are shown in FIG. 13D for a short excerpt. To make the two comparable, the random starting phase was subtracted from the corrected phase.

FIGS. 13A-13D show the mis-synchronization due to the usage of two DAQ boards. FIG. 13A shows the raw phase angles obtained by CS recalibration over time and FIG. 13B shows their histogram. A fixed phase jump can be observed in FIG. 13A, and the histogram shown in FIG. 13B shows two distinct peak with similar distribution. This phase jump can be caused by the one pixel shift during the simultaneous triggering of the two DAQ boards. The numerically corrected phase angles (e.g., CS-based calibration) are plotted against that of FIG. 13C pre-measured calibration and FIG. 13D full calibration. The CS-based calibration provides a significantly superior phase stability to that of the pre-measured calibration and a comparable result to that of the full calibration.

### EXEMPLARY CONCLUSION

By exploiting the redundancy/sparsity existed in the reference clock signal, only a small portion of the data is needed to accurately reconstruct the original signal, which can greatly reduce the system's workload on data transfer and storage. The quality of the reconstructed intensity profile and phase profile, were evaluated. Less than 50% of the original data can be needed to reconstruct the original signal with errors lower than the noise level. Table 1 below illustrated examples of the effective compression achieved using the exemplary system, method, and computer-accessible medium, and apparatus.

TABLE 1

| A-line rate of | Sampling points | | | 1/Compression | Data rate after |
|---|---|---|---|---|---|
| the source | per A-line | Bit depth | Data rate | ratio | compression |
| 200 kHz | 2000 | 12-bit | 1.6 GB/s | 30% | 1.04 GB/s |
| 200 kHz | 8000 | 12-bit | 6.4 GB/s | 5% | 3.36 GB/s |
| 1.5 MHz | 1000 | 12-bit | 12 GB/s | 30% | 7.8 GB/s |
| 1.5 MHz | 2000 | 12-bit | 24 GB/s | 5% | 12.6 GB/s |

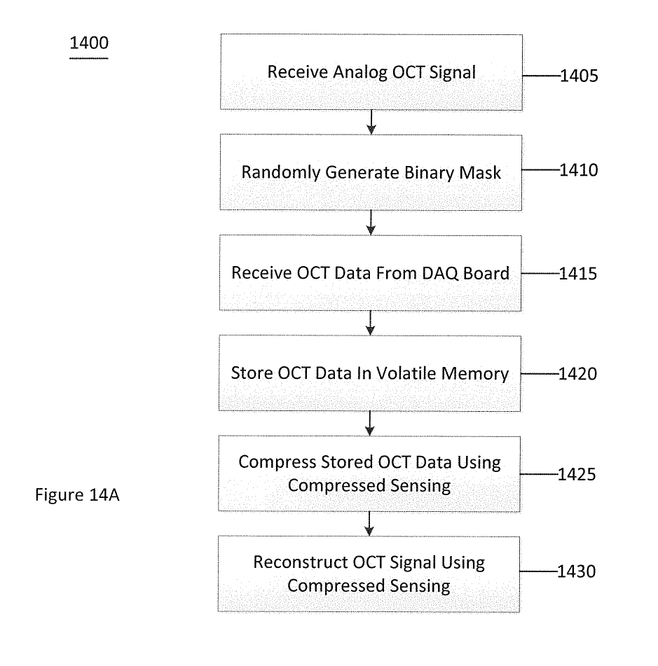

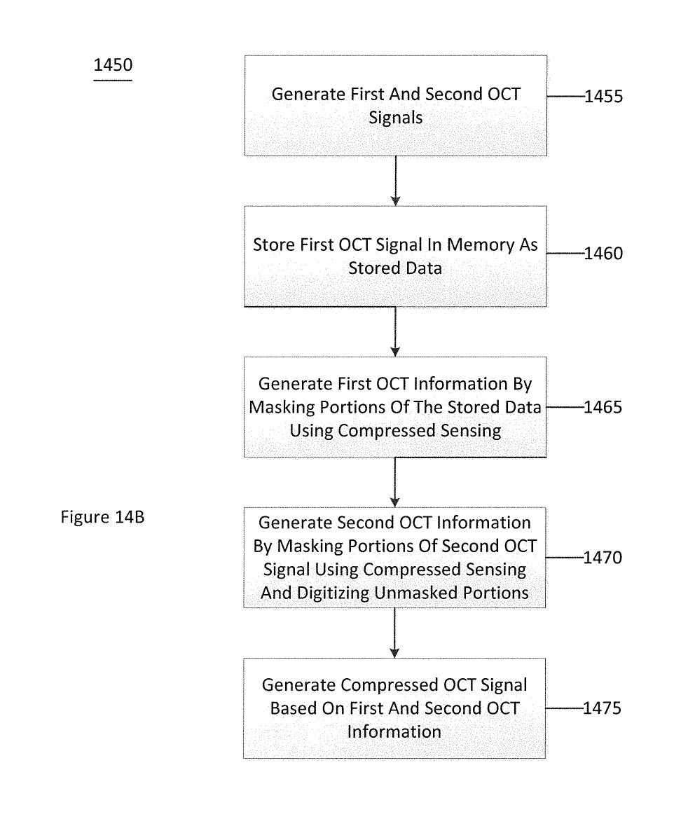

FIG. 14A shows an exemplary flow diagram of an exemplary method 1400 for compressing data that is based on the optical coherence tomography signal according to an exemplary embodiment of the present disclosure. For example, at procedure 1405, an analog OCT signal can be received. At procedure 1410, a binary mask can be randomly generated. At procedure 1415, OCT data from a DAQ board, which can be used to digitize the OCT signal, can be received, and the data can be stored in a volatile memory at procedure 1420. At procedure 1425, the stored OCT data can be compressed using a compressed sensing procedure. At procedure 1430, the OCT signal can be reconstructed using the same or a different compressed sensing procedure.

FIG. 14B shows an exemplary flow diagram of an exemplary method 1450 for compressing the optical coherence tomography signal according to an exemplary embodiment of the present disclosure. For example, at procedure 1455, first and second OCT signals can be generated. At procedure 1460, the first OCT signal can be stored in memory as stored data. At procedure 1465, first OCT information can be generated by masking portions of the stored data using a compressed sensing procedure. At procedure 1470, second OCT information can be generated by masking portions of the second OCT using the same or a different compressed sensing procedure, and the unmasked portions can be digitized. At procedure 1475, the compressed OCT signal can be generated based on the first and second OCT information.

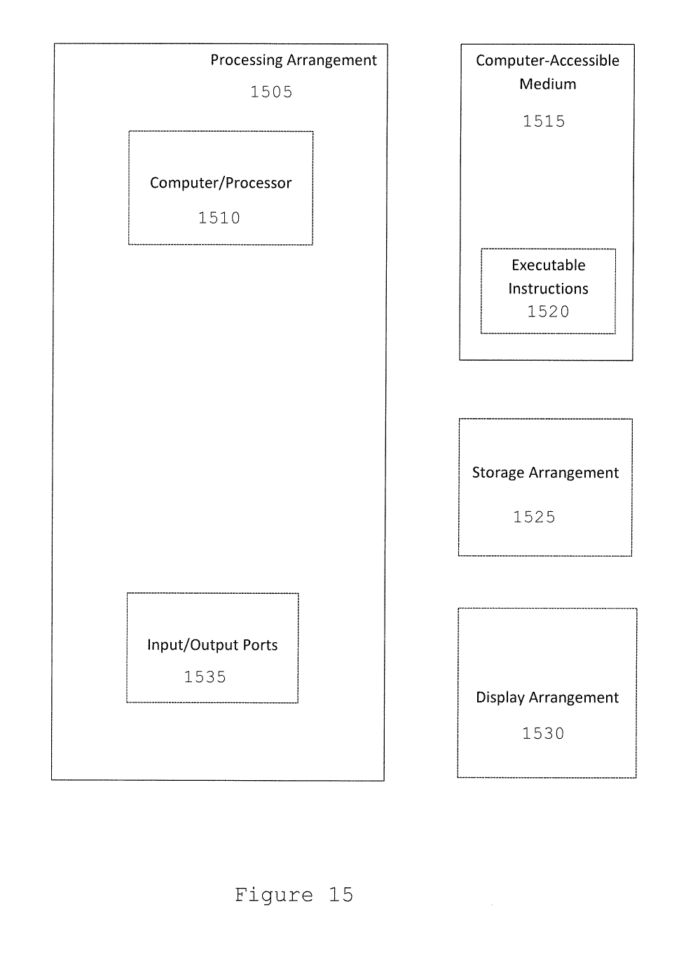

FIG. 15 shows a block diagram of an exemplary embodiment of a system according to the present disclosure. For example, exemplary procedures in accordance with the present disclosure described herein can be performed by a processing arrangement and/or a computing arrangement 1505. Such processing/computing arrangement 1505 can be, for example entirely or a part of, or include, but not limited to, a computer/processor 1510 that can include, for example one or more microprocessors, and use instructions stored on a computer-accessible medium (e.g., RAM, ROM, hard drive, or other storage device).

As shown in FIG. 15, for example a computer-accessible medium 1515 (e.g., as described herein above, a storage device such as a hard disk, floppy disk, memory stick, CD-ROM, RAM, ROM, etc., or a collection thereof) can be provided (e.g., in communication with the processing arrangement 1505). The computer-accessible medium 1515 can contain executable instructions 1520 thereon. In addition or alternatively, a storage arrangement 1525 can be provided separately from the computer-accessible medium 1515, which can provide the instructions to the processing arrangement 1505 so as to configure the processing arrangement to execute certain exemplary procedures, processes, and methods, as described herein above, for example.

Further, the exemplary processing arrangement 1505 can be provided with or include an input/output arrangement 1535, which can include, for example a wired network, a wireless network, the internet, an intranet, a data collection probe, a sensor, etc. As shown in FIG. 15, the exemplary processing arrangement 1505 can be in communication with an exemplary display arrangement 1530, which, according to certain exemplary embodiments of the present disclosure, can be a touch-screen configured for inputting information to the processing arrangement in addition to outputting information from the processing arrangement, for example. Further, the exemplary display 1530 and/or a storage arrangement 1525 can be used to display and/or store data in a user-accessible format and/or user-readable format.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification, drawings and claims thereof, can be used synonymously in certain instances, including, but not limited to, for example, data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

### EXEMPLARY REFERENCES

The following references are hereby incorporated by reference in their entireties:
[1] B. Golubovic et al, Opt. Lett. 22(22), 1704-1706 (1997).
[2] X. Wei et al, Opt. Express 6, 3855-3864 (2015)
[3] W. Wieser et al, Opt. Express 5, 2963-2977 (2014).
[4] H. Y. Lee et al, PNAS 2015 112 (10) 3128-3133.
[5] S. Song et al, Appl. Phys. Lett. 108:19 (2016)
[6] M. Gora et al, Opt. Express 17, 14880-14894 (2009)
[7] Y. Ling et al, Optics Letter, 42, 1333-1336 (2017)
[8] E. Candès et al, IEEE Trans. Inf. Theory, 52 (2), 489-509 (2006).
[9] D. Donoho, IEEE Trans. Inf. Theory, 52 (4), 1289-1306 (2006).
[10] II. Y. Lee, P. D. Raphael, J. Park, A. K. Ellerbee, B. E. Applegate, and J. S. Oghalai, “Noninvasive in vivo imaging reveals differences between tectorial membrane and basilar membrane traveling waves in the mouse cochlea,” Proc. Natl. Acad. Sci. 112, 3128-3133 (2015).
[11] R. K. Wang, S. L. Jacques, Z. Ma, S. Hurst, S. R. Hanson, and A. Gruber, “Three dimensional optical angiography,” Opt. Express 15, 4083-4097 (2007).
[12] D. M. Schwartz, J. Fingler, D. Y. Kim, R. J. Zawadzki, L. S. Morse, S. S. Park, S. E. Fraser, and J. S. Werner, “Phase-variance optical coherence tomography: A technique for noninvasive angiography,” Ophthalmology 121, 180-187 (2014).
[13] A. Zhang, Q. Zhang, C.-L. Chen, and R. K. Wang, “Methods and algorithms for optical coherence tomography-based angiography: a review and comparison,” J. Biomed. Opt. 20, 100901 (2015).
[14] Y. Zhao, Z. Chen, C. Saxer, S. Xiang, J. F. de Boer, and J. S. Nelson, “Phase-resolved optical coherence tomography and optical Doppler tomography for imaging blood flow in human skin with fast scanning speed and high velocity sensitivity.” Opt. Lett. 25, 114-116 (2000).
[15] R. A. Leitgeb, R. M. Werkmeister, C. Blatter, and L. Schmetterer, “Doppler optical coherence tomography,” Prog. Retin. Eye Res. 41, 26-43 (2014).
[16] S. Wang and K. V. Larin, “Shear wave imaging optical coherence tomography (SWI-OCT) for ocular tissue biomechanics,” Opt. Lett. 39, 41-44 (2014).
[17] S. Wang and K. V. Larin, “Optical coherence elastography for tissue characterization: A review,” J. Biophotonics 8, 279-302 (2015).
[18] X. Liang, A. L. Oldenburg, V. Crecea, E. J. Chaney, and S. A. Boppart, “Optical micro-scale mapping of dynamic biomechanical tissue properties.” Opt. Express 16, 11052-11065 (2008).
[19] R. F. Spaide, J. G. Fujimoto, and N. K. Waheed, “Image artifacts in optical coherence tomography angiography,” Retina 35, 2163-2180 (2015).
[20] L. An and R. K. Wang, “In vivo volumetric imaging of vascular perfusion within human retina and choroids with optical micro-angiography,” Opt. Express 16, 11438-11452 (2008).
[21] Y. K. Tao, A. M. Davis, and J. A. Izatt, “Single-pass volumetric bidirectional blood flow imaging spectral domain optical coherence tomography using a modified Hilbert transform,” Opt. Express 16, 12350-12361 (2008).
[22] W. Wieser, W. Draxinger, T. Klein, S. Karpf, T. Pfeiffer, and R. Huber, “High definition live 3D-OCT in vivo: design and evaluation of a 4D OCT engine with 1 GVoxel/s,” Biomed. Opt. Express 5, 2963-2977 (2014).
[23] S. Song, W. Wei, B. Y. Hsieh, I. Pelivanov, T. T. Shen, M. O'Donnell, and R. K. Wang, “Strategies to improve phase-stability of ultrafast swept source optical coherence tomography for single shot imaging of transient mechanical waves at 16 kHz frame rate,” Appl. Phys. Lett. 108, 191104 (2016).
[24] T. Klein and R. Huber, “High-speed OCT light sources and systems [Invited],” Biomed. Opt. Express 8, 828-859 (2017).
[25] B. Golubovic, B. E. Bouma, G. J. Tearney, and J. G. Fujimoto, “Optical frequency-domain reflectometry using rapid wavelength tuning of a Cr4+:forsterite laser.” Opt. Lett. 22, 1704-1706 (1997).
[26] R. A. Huber, M. Wojtkowski, and J. G. Fujimoto, “Fourier Domain Mode Locking (FDML): A new laser operating regime and applications for optical coherence tomography.” Opt. Express 14, 3225-3237 (2006).
[27] S. Yun, G. Tearney, J. de Boer, N. Iftimia, and B. Bouma, “High-speed optical frequency-domain imaging,” Opt. Express 11, 2953-2963 (2003).
[28] W. Wieser, B. R. Biedermann, T. Klein, C. M. Eigenwillig, and R. Huber, “Multi-Megahertz OCT: High quality 3D imaging at 20 million A-scans and 4.5 GVoxels per second,” Opt. Express 18, 14685-14704 (2010).
[29] I. Grulkowski, J. J. Liu, B. Potsaid, V. Jayaraman, C. D. Lu, J. Jiang, A. E. Cable, J. S. Duker, and J. G. Fujimoto, “Retinal, anterior segment and full eye imaging using ultrahigh speed swept source OCT with vertical-cavity surface emitting lasers,” Biomed. Opt. Express 3, 2733-2751 (2012).
[30] X. Wei, A. K. S. Lau, Y. Xu, K. K. Tsia, and K. K. Y. Wong, “28 MHz swept source at 1.0 μm for ultrafast quantitative phase imaging,” Biomed. Opt. Express pp. 3855-3864 (2015).
[31] Y. Ling, Y. Gan, X. Yao, and C. P. Hendon, “Phase-noise analysis of swept-source optical coherence tomography systems,” Opt. Lett. 42, 1333-1336 (2017).
[32] Y. Yasuno, V. D. Madjarova, S. Makita, M. Akiba, A. Morosawa, C. Chong, T. Sakai, K.-P. Chan, M. Itoh, and T. Yatagai, “Three-dimensional and high-speed swept-source optical coherence tomography for in vivo investigation of human anterior eye segments.” Opt. Express 13, 10652-10664 (2005).
[33] M. Gora, K. Karnowski, M. Szkulmowski, B. J. Kaluzny, R. Huber, A. Kowalczyk, and M. Wojtkowski, “Ultra highspeed swept source OCT imaging of the anterior segment of human eye at 200 kHz with adjustable imaging range.” Opt. Express 17, 14880-14894 (2009).
[34] Z. Shangguan, Y. Shen, P. Li, and Z. Ding, “Wavenumber calibration and phase measurement in swept source optical coherence tomography (in Chinese),” Acta Phys. Sinica 65, 034201 (2016).
[35] M. Singh, C. Wu, C.-H. Liu, J. Li, A. Schill, A. Nair, and K. V. Larin, “Phase-sensitive optical coherence elastography at 1.5 million A-Lines per second,” Opt. Lett. 40, 2588-2591 (2015).
[36] S. Amarakoon, J. H. De Jong, B. Braaf, S. Yzer, T. Missotten, M. E. Van Velthoven, and J. F. De Boer, “Phase-resolved doppler optical coherence tomographic features in retinal angiomatous proliferation,” Am. J. Ophthalmol. 160, 1044-1054 (2015).
[37] S. Kim, P. D. Raphael, J. S. Oghalai, and B. E. Applegate, “High-speed spectral calibration by complex FIR filter in phase-sensitive optical coherence tomography,” Biomed. Opt. Express 7, 1430-1444 (2016).
[38] W. Chen, C. Du, and Y. Pan, “Cerebral capillary flow imaging by wavelength-division-multiplexing swept-source optical Doppler tomography,” (2018).
[39] E. Candès, J. Romberg, and T. Tao, “Robust uncertainty principles: Exact signal reconstruction from highly incomplete frequency information,” IEEE Transactions on Inf. Theory 52, 489-509 (2006).
[40] D. Donoho, “Compressed sensing,” IEEE Transactions on Inf. Theory 52, 1289-1306 (2006).
[41] N. Mohan, I. Stojanovic, W. C. Karl, B. E. A. Saleh, and M. C. Teich, “Compressed sensing in optical coherence tomography,” in Proc. SPIE 7570, (2010), p. 75700L.
[42] X. Liu and J. U. Kang, “Compressive SD-OCT: the application of compressed sensing in spectral domain optical coherence tomography,” Opt. Express 18, 22010-22019 (2010).
[43] E. Lebed, P. J. Mackenzie, M. V. Sarunic, and M. F. Beg, “Rapid volumetric OCT image acquisition using Compressive Sampling,” Opt. Express 18, 21003-21012 (2010).
[44] W. Meiniel, Y. Gan, C. P. Hendon, J.-C. Olivo-Marin, A. Laine, and E. D. Angelini, “Sparsity-based simplification of spectral-domain optical coherence tomography images of cardiac samples,” in Biomedical Imaging (ISBI), 2016 IEEE 13th International Symposium on, (IEEE, 2016), pp. 373-376.
[45] W. Meiniel, J.-C. Olivo-Marin, and E. Angelini, “A sparsity-based simplification method for segmentation of spectral-domain optical coherence tomography images,” in Wavelets and Sparsity XVII, vol. 10394 (International Society for Optics and Photonics, 2017), p. 1039406.
[46] E. Candes and J. Romberg, “Sparsity and incoherence in compressive sampling,” Inverse problems 23, 969 (2007).
[47] D. L. Donoho and X. Huo, “Uncertainty principles and ideal atomic decomposition,” IEEE transactions on information theory 47, 2845-2862 (2001).
[48] M. Lustig, D. Donoho, and J. M. Pauly, “Sparse MRI: The application of compressed sensing for rapid MR imaging,” Magn. Reson. Medicine 58, 1182-1195 (2007).
[49] J. Bobin, J.-L. Starck, and R. Ottensamer, “Compressed sensing in astronomy,” IEEE J. Sel. Top. Signal Process. 2, 718-726 (2008).
[50] M. A. Herman and T. Strohmer, “High-resolution radar via compressed sensing,” IEEE Transactions on Signal Process. 57, 2275-2284 (2009).
[51] M. M. Marim, M. Atlan, E. Angelini, and J.-C. OlivoMarin, “Compressed sensing with off-axis frequency-shifting holography,” Opt. Left. 35, 871-873 (2010).
[52] M. F. Duarte and R. G. Baraniuk, “Spectral compressive sensing,” Appl. Comput. Harmon. Analysis 35, 111-129 (2013).
[53] R. Baraniuk, V. Cevher, M. Duarte, and C. Hegde, “Model-based compressive sensing,” IEEE Transactions on Inf. Theory 56, 1982-2001 (2010).
[54] S. Becker, J. Bobin, and E. Candès, “NESTA: A Fast and Accurate First-Order Method for Sparse Recovery,” J. on Imaging Sci. 4, 1-39 (2011).
[55] Y. Ling, X. Yao, and C. P. Hendon, “Highly phase-stable 200 kHz swept-source optical coherence tomography based on KTN electro-optic deflector,” Biomed. Opt. Express 8, 3687-3699 (2017).
[56] R. K. Wang and L. An, “Doppler optical micro-angiography for volumetric imaging of vascular perfusion in vivo,” Opt. Express 17, 8926-8940 (2009).
[57] D. Xu, Y. Huang, and J. U. Kang, “Real-time compressive sensing spectral domain optical coherence tomography,” Opt. Lett. 39, 76-79 (2014).

What is claimed is:

1. A non-transitory computer-accessible medium having stored thereon computer-executable instructions for compressing data that is based on an optical coherence tomography (OCT) signal, wherein, when a computer hardware arrangement executes the instructions, the computer hardware arrangement is configured to perform procedures comprising:
receiving OCT data from at least one digital acquisition (DAQ) board that is based on the OCT signal;
storing the OCT data in a volatile memory; and
compressing the stored OCT data using a compressed sensing (CS) procedure.

2. The computer-accessible medium of claim 1, wherein the CS procedure is based on a software mask residing on the computer hardware arrangement.

3. The computer-accessible medium of claim 2, wherein the computer hardware arrangement is configured to compress the stored OCT data using the software mask to mask particular portions of the stored OCT data.

4. The computer-accessible medium of claim 3, wherein the computer hardware arrangement is further configured to store the compressed OCT data in a non-volatile data storage arrangement.

5. The computer-accessible medium of claim 1, wherein the OCT signal is an OCT calibration signal.

6. The computer-accessible medium of claim 1, wherein the computer hardware arrangement is further configured to reconstruct the OCT data using the CS procedure.

7. The computer-accessible medium of claim 1, wherein the computer hardware arrangement is further configured to receive an analog signal related to the OCT data, and generate the OCT data using the at least one DAQ board based on the analog signal.

8. The computer-accessible medium of claim 1, wherein the computer hardware arrangement is configured to compress the stored OCT data by down-sampling the OCT data using the CS procedure.

9. The computer-accessible medium of claim 1, wherein the CS procedure is based on a binary mask having a particular compression ratio.

10. The computer-accessible medium of claim 9, wherein the computer hardware arrangement is further configured to randomly generate the binary mask.

11. The computer-accessible medium of claim 1, wherein the computer hardware arrangement is further configured to generate the OCT data by fully digitizing a sample channel and a clock channel from an OCT scan at a full rate.

12. The computer-accessible medium of claim 12, wherein the computer hardware arrangement is further configured to (i) fully digitize the sample channel using a first DAQ Board, and (ii) fully digitize the clock channel using a second DAQ board, wherein the first DAQ board is different from the second DAQ board.

13. The computer-accessible medium of claim 1, wherein the computer hardware arrangement is configured to generate the OCT data by digitizing an OCT signal related to the OCT data using a plurality of registers, wherein each of the registers specifies a trigger event to ignore a portion of the OCT signal during digitization.

14. A digital acquisition (DAQ) board for use in an optical coherence tomography (OCT) system, comprising:
at least one hardware signal mask configured to receive an OCT signal and mask particular portions of the OCT signal to generate a masked OCT signal; and
at least one analog to digital (A/D) converter receiving and converting the unmasked OCT signal into a digital format.

15. The DAQ board of claim 14, wherein the OCT signal is an OCT calibration signal.

16. The DAQ board of claim 14, wherein the at least one hardware signal mask is configured to mask the particular portions based on a compressed sensing procedure.

17. The DAQ board of claim 14, wherein the at least one hardware signal mask is configured to mask the particular portions using a plurality of registers, wherein each of the registers specifies a trigger event to ignore at least one portion of the OCT signal during digitization.

18. A method for compressing an optical coherence tomography (OCT) signal, comprising:
generating first and second OCT signals based on the OCT signal;
storing the first OCT signal in memory as stored data;
masking particular portions of the stored data using a first compressed sensing (CS) procedure thereby generating first OCT information;
generating second OCT information by:
masking particular portions of the second OCT signal based on a second CS procedure; and
digitizing unmasked portions of the second OCT signal; and
using a computer hardware arrangement, generating the compressed OCT signal based on the first OCT information and the second OCT information.

19. The method of claim 18, wherein the first OCT signal is a calibration signal and the second OCT signal is a sample signal.

20. The method of claim 18, further comprising masking the particular portions of the stored data based on a chirped sine function sampled over a particular number of data points.
